(19) United States
(12) Patent Application Publication
(10) Pub. No.: US 2024/0207424 A1
(43) Pub. Date: Jun. 27, 2024

(54) USE OF HER2-TARGETING ANTIBODY-DRUG CONJUGATE IN TREATMENT OF HER2-LOW EXPRESSING BREAST CANCER

(71) Applicant: REMEGEN CO., LTD., Yantai (CN)

(72) Inventors: Jianmin FANG, Yantai (CN); Xiaohong SU, Yantai (CN)

(21) Appl. No.: 18/511,869

(22) Filed: Nov. 16, 2023

Related U.S. Application Data
(63) Continuation of application No. PCT/CN22/93632, filed on May 18, 2023.

(30) Foreign Application Priority Data
May 24, 2021 (CN) 202110565350.2

(51) Int. Cl. A61K 47/68; A61P 35/00; C07K 16/32

(52) U.S. Cl. CPC A61K 47/68031; A61K 47/6889; A61P 35/00; C07K 16/32

(57) ABSTRACT

Provided is a method for treating a patient with Human Epidermal Growth Factor Receptor 2 (HER2)-low expressing breast cancer.

### CROSS REFERENCE TO RELATED APPLICATIONS

This application claims the priority benefit of Chinese Application No. 202110565350.2, filed May 24, 2021, which is incorporated herein by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 761682008741SEQLIST.txt, date recorded: May 16, 2022, size: 9,994 bytes).

### FIELD

The present disclosure relates to the field of treatment of HER2-low expressing breast cancer, and to use of a Human Epidermal Growth Factor Receptor 2 (HER2)-targeting antibody-drug conjugate in the treatment of patients with HER2-low expressing breast cancer.

### BACKGROUND

Human Epidermal Growth Factor Receptor 2 (HER2), also known as ERBB-2, or proto-oncogene Neu, is a tyrosine protein kinase receptor encoded by the ERBB2 (HER2) gene on chromosome 17q12 (Moasser M. M. The oncogene HER2: Its signaling and transforming functions and its role in human cancer pathogenesis. Oncogene. 2007; 26: 6469-6487). In addition to Epidermal Growth Factor Receptor (EGFR, ERBB-1), Human Epidermal Growth Factor Receptor 3 (HER3, ERBB-3), and Human Epidermal Growth Factor Receptor 4 (HER4, ERBB-4), HER2 is also a member of the epidermal growth factor receptor family. Since the HER2 protein has no extracellular region for ligand binding, no growth factors can bind to it directly. However, it can form a heterodimer with a ligand-binding member of the EGF receptor family, thereby enhancing kinase-mediated downstream signal (Iqbal N., Iqbal N. Human epidermal growth factor receptor 2 (HER2) in cancers: Overexpression and therapeutic implications. Mol. Biol. Int. 2014: 852748).

HER2 is expressed on epithelial cell membranes of the gastrointestinal tract, respiratory tract, reproductive tract, urinary tract, skin, breast, placenta, etc., as well as on cardiac and skeletal muscle cells (Uhlen M et al. Proteomics. Tissue-based map of the human proteome. Science. 2015; 347:1260419). In fetal tissues, the expression level of HER2 is generally higher than that in the corresponding normal adult tissues (Press M. F. et al. Expression of the HER-2/neu proto-oncogene in normal human adult and fetal tissues. Oncogene. 1990 5(7): 953-62). Overexpression of HER2 can promote tumorigenesis through various mechanisms, such as breast cancer, gastric cancer, and lung cancer.

Breast cancer is a common malignant tumor in women. Due to changes in people's lifestyle concepts and ecological environment, the incidence of breast cancer is also increasing significantly. According to current treatment guidelines, breast cancer is generally classified as HER2-positive or HER2-negative. HER2-positive generally refers to IHC 3+ or IHC 2+/FISH+ (IHC: immunohistochemistry detection; FISH: fluorescence in situ hybridization detection). In addition, there are HER2-low expressing patients (IHC 2+/FISH negative or IHC1+) (metastatictrialtalk.org/research-news/HER2-low-expressing-a-new-subcategory-of-HER2-negative-breast-cancer/). According to clinical statistics, more than 50% of breast cancer may be breast cancer with low HER2 expression level (Tarantino P et al. HER2-low breast cancer: pathological and clinical landscape. J Clin Oncol. 2020; 38(17): 1951-1962. doi: 10.1200/JCO.19.02488, Wolff A. C. et al. Human epidermal growth factor receptor 2 testing in breast cancer: American society of clinical oncology/college of american pathologists clinical practice guideline focused update. J. Clin. Oncol. 2018; 36: 2105-2122. doi: 10.1200/JCO.2018.77.8738).

Antibody-Drug Conjugates (ADCs) are molecules that are formed by covalently binding monoclonal antibodies to cytotoxic drugs through a linkage unit. After the antibody binds to a specific antigen on the surface of the cancer cell, the cytotoxic drug is released into the cell to exert its effect. Using cleavable linkage units, ADCs can be engineered to be released from target cells into the extracellular space, so that surrounding and bystander cells, which may or may not express the ADC target antigen, can be killed by uptake of cytotoxic drugs (Beck A. et al. Strategies and challenges for the next generation of antibody-drug conjugates. Nat. Rev. Drug Discov. 2017; 16: 315-337, Staudacher A. H., Brown M. P. Antibody drug conjugates and bystander killing: Is antigen-dependent internalisation required? Br. J. Cancer. 2017; 117: 1736-1742).

At present, a variety of antibody-drug conjugates targeting HER-2 have been used in clinical studies of breast cancer (see Table 1).

TABLE 1
HER2-targeting ADCs.

| | Global highest | Original |
|---|---|---|
| Drug Name | R&D status | R&D company |
| Fam-trastuzumab | Approved for | Daiichi Sankyo Co., Ltd. |
| deruxtecan | launch (2019) | |
| Ado-trastuzumab | Approved for | Genentech |
| emtansine | launch (2013) | |
| Disitamab Vedotin | Applying to | RemeGen Co., Ltd. |
| | Launch | |
| TAA-013 | Phase 3 clinical | TOT Biopharm Co., Ltd. |
| Trastuzumab | Phase 3 clinical | Synthon |
| duocarmazine | | |
| BAT-8001 | Phase 3 clinical | Bio-Thera |
| ARX-788 | Phase 3 clinical | Ambrx |
| Patritumab Deruxtecan | Phase 2 clinical | Daiichi Sankyo Co., Ltd. |
| MRG-002 | Phase 2 clinical | Shanghai Miracogen Inc. |
| A-166 | Phase 2 clinical | Sichuan Kelun |
| | | Pharmaceutical Co., Ltd. |
| Trastuzumab conjugate | Phase 1 clinical | Biointegrator |
| Anti-HER2 ADC | Phase 1 clinical | Pfizer |
| GQ-1001 | Phase 1 clinical | |
| Disitamab Vedotin | Phase 1 clinical | CSPC Zhongqi |
| | | Pharmaceutical Technology |
| | | (Shijiazhuang) Co., Ltd. |
| ZW-49 | Phase 1 clinical | Zymeworks Inc |
| Recombinant | Phase 1 clinical | Qilu Pharmaceutical |
| anti-HER2 | | Co., Ltd. |
| humanized monoclonal | | |
| antibody-DM1 | | |
| ALT-P7 | Phase 1 clinical | Alteogen |
| GB-251 | Phase 1 clinical | Genor Biopharma Co., Ltd. |
| LCB14-0110 | Phase 1 clinical | Legochembio |
| SHR-A1201 | Phase 1 clinical | Jiangsu Hengrui |
| | | Pharmaceuticals Co., Ltd. |
| Recombinant | Phase 1 clinical | Hangzhou DAC |
| anti-HER2 | | Biotechnology Co., Ltd. |
| antibody-Tub114 | | |
| Anti-HER2 monoclonal | Phase 1 clinical | Shanghai Jiaolian Drug |
| antibody-MCC-DM1 | | Research and Development |
| conjugate | | Co., Ltd.; |
| | | Shanghai Pharmaceuticals |
| | | Holding Co., Ltd. |
| RG-6148 | Phase 1 clinical | Roche |

However, because the currently marketed drugs targeting HER2 are all aimed at HER2-positive patients, they cannot be effectively used to treat HER2-low expressing patients (IHC 2+/FISH negative or IHC1+).

From data disclosed in clinical information, only HER2-low expressing advanced or metastatic breast cancer patients treated with DS-8201 had positive therapeutic effects, where the objective remission rate (ORR) was 37.0%, the median duration of response was 10.4 months, the median progression-free survival was 11.1 months, and the median overall survival was 29.4 months (95% CI, 12.9-29.4) (www.onclive.com/view/trastuzumab-deruxtecan-is-active-in-HER2-low-expressing-breast-cancer).

Thus, there is a need in the art for compositions, such as anti-HER2 antibody drug conjugates, uses of such compositions and methods for treating HER2-low expressing breast cancer.

All references cited herein, including patent applications, patent publications, and UniProtKB/Swiss-Prot Accession numbers are herein incorporated by reference in their entirety, as if each individual reference were specifically and individually indicated to be incorporated by reference.

### SUMMARY

The present disclosure provides methods and uses for treating HER2-low expressing breast cancer patients with an anti-HER2 antibody-drug conjugate (ADC). These methods and uses were based at least in part on an in-depth analysis of a large number of clinical data. The present disclosure surprisingly found that an ADC produced unexpected technical effects in the treatment of HER2-low expressing breast cancer patients. Specifically, RC48-ADC showed consistent therapeutic efficacy in HER2-positive and HER2-low expressing subgroups of patients.

In one aspect, provided herein is a use of an antibody-drug conjugate (ADC) in the preparation of a medicine for treating of a patient with Human Epidermal Growth Factor Receptor 2 (HER2)-low expressing breast cancer, wherein the ADC has the structure of the general formula Ab-(L-U)ₙ, wherein: Ab represents an anti-HER2 antibody, L represents a linker, U represents a conjugated cytotoxic molecule, and n is an integer from 1 to 8 and represents the number of cytotoxic molecules bound to each antibody; wherein the antibody comprises a heavy chain variable region and a light chain variable region, wherein the CDR sequences of the heavy chain variable region and/or the CDR sequences of the light chain variable region have the same CDR sequences as Disitamab vedotin; wherein the linker L comprises Maleimido-Caproyl-Valine-Citrulline-p-Aminobenzyloxy (mc-vc-pAB), wherein the linker is covalently linked to the anti-HER2 antibody by means of sulfhydryl conjugation, and the linking site is the interchain disulfide bond site of the anti-HER2 antibody; and wherein the cytotoxic molecule U comprises MMAE (monomethyl auristatin E).

In another aspect, provided herein is a method for treating a patient with Human Epidermal Growth Factor Receptor 2 (HER2)-low expressing breast cancer, comprising administering to the patient a therapeutically effective amount of an antibody-drug conjugate (ADC), wherein the ADC has the structure of the general formula Ab-(L-U)ₙ, wherein: Ab represents an anti-HER2 antibody, L represents a linker, U represents a conjugated cytotoxic molecule, and n is an integer from 1 to 8 and represents the number of cytotoxic molecules bound to each antibody; wherein the antibody comprises a heavy chain variable region and a light chain variable region, wherein the CDR sequences of the heavy chain variable region and/or the CDR sequences of the light chain variable region have the same CDR sequences as Disitamab vedotin, wherein the linker L comprises Maleimido-Caproyl-Valine-Citrulline-p-Aminobenzyloxy (mc-vc-pAB), wherein the linker is covalently linked to the anti-HER2 antibody by means of sulfhydryl conjugation, and the linking site is the interchain disulfide bond site of the anti-HER2 antibody; and wherein the cytotoxic molecule U comprises MMAE (monomethyl auristatin E).

In some embodiments, which may be combined with any of the preceding aspects, the HER2-low expressing breast cancer patient is a patient whose HER2 is detected as immunohistochemistry (IHC) 2+/fluorescence in situ hybridization (FISH) negative or IHC1+. In some embodiments, which may be combined with any of the preceding aspects or embodiments, HER2 is detected as IHC 2+/FISH negative or IHC1+ in a sample from the breast cancer. In some embodiments, which may be combined with any of the preceding aspects or embodiments, HER2 is detected using an immunohistochemistry (IHC) assay and/or a fluorescence in situ hybridization (FISH) assay.

In some embodiments, which may be combined with any of the preceding aspects or embodiments, the anti-HER2 antibody is a murine, chimeric, humanized or fully human antibody. In some embodiments, which may be combined with any of the preceding aspects or embodiments, the anti-HER2 antibody is of the IgG class. In some embodiments, the anti-HER2 antibody has an IgG1, IgG2, or IgG4 isotype.

In some embodiments, which may be combined with any of the preceding aspects or embodiments, the anti-HER2 antibody comprises a heavy chain variable region (VH) and a light chain variable region (VL), wherein: (a) the VH comprises a CDR-H1 comprising the amino acid sequence GYTFTDYY (SEQ ID NO:3), a CDR-H2 comprising the amino acid sequence VNPDHGDS (SEQ ID NO:4), and a CDR-H3 comprising the amino acid sequence ARNYLFDH (SEQ ID NO:5), and (b) the VL comprises a CDR-L1 comprising the amino acid sequence QDVGTA (SEQ ID NO:6), a CDR-L2 comprising the amino acid sequence WAS (SEQ ID NO:7), and a CDR-L3 comprising the amino acid sequence HQFATYT (SEQ ID NO:8).

In some embodiments, which may be combined with any of the preceding aspects or embodiments, the anti-HER2 antibody comprises a heavy chain variable region (VH) and a light chain variable region (VL), wherein: (a) the VH comprises a CDR-H1 comprising the amino acid sequence DYYIH (SEQ ID NO: 11), a CDR-H2 comprising the amino acid sequence RVNPDHGDSYYNQKFKD (SEQ ID NO: 12), and a CDR-H3 comprising the amino acid sequence ARNYLFDHW (SEQ ID NO: 13), and (b) the VL comprises a CDR-L1 comprising the amino acid sequence KASQDVGTAVA (SEQ ID NO: 14), a CDR-L2 comprising the amino acid sequence WASIRHT (SEQ ID NO: 15), and a CDR-L3 comprising the amino acid sequence HQFATYT (SEQ ID NO: 16).

In some embodiments, which may be combined with any of the preceding aspects or embodiments, the anti-HER2 antibody comprises a heavy chain variable region (VH) comprising the amino acid sequence of SEQ ID NO. 9, and a light chain variable region (VL) comprising the amino acid sequence of SEQ ID NO: 10.

In some embodiments, which may be combined with any of the preceding aspects or embodiments, the anti-HER2 antibody is a human IgG antibody. In some embodiments, which may be combined with any of the preceding aspects or embodiments, the anti-HER2 antibody is a human IgG1, IgG2, IgG3, or IgG4 antibody.

In some embodiments, which may be combined with any of the preceding aspects or embodiments, the amino acid sequence of the heavy chain of the antibody is shown in SEQ ID NO:1, and the amino acid sequence of the light chain of the antibody is shown in SEQ ID NO:2.

In some embodiments, which may be combined with any of the preceding aspects or embodiments, the ADC is Disitamab vedotin or a biosimilar thereof.

In some embodiments, which may be combined with any of the preceding aspects or embodiments, the average Drug-to-Antibody Ratio (DAR) value of the ADC is any number from 2 to 7. In some embodiments, which may be combined with any of the preceding aspects or embodiments, the average DAR value is 4±0.5.

In some embodiments, which may be combined with any of the preceding aspects or embodiments, the breast cancer is infiltrating locally advanced or metastatic breast cancer as established by histology and/or cytology, and is unresectable.

In some embodiments, which may be combined with any of the preceding aspects or embodiments, the patient has previously received one or more prior treatments. In some embodiments, the one or more prior treatments are selected from a chemotherapy drug, a targeted therapy, an immunotherapy and an endocrine therapy. In some embodiments, which may be combined with any of the preceding aspects or embodiments, the patient has previously received taxane systemic therapy. In some embodiments, which may be combined with any of the preceding aspects or embodiments, the patient has previously received systemic therapy with trastuzumab or a biosimilar thereof at least once.

In some embodiments, which may be combined with any of the preceding aspects or embodiments, the medicine or the ADC is administered intranasally, subcutaneously, intradermally, intramuscularly or intravenously.

In some embodiments, which may be combined with any of the preceding aspects or embodiments, the ADC is administered at a dose of 2.0 mg/kg every 2 weeks.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIG. 1 is a schematic diagram of the structure of monomethyl auristatin E (MMAE).

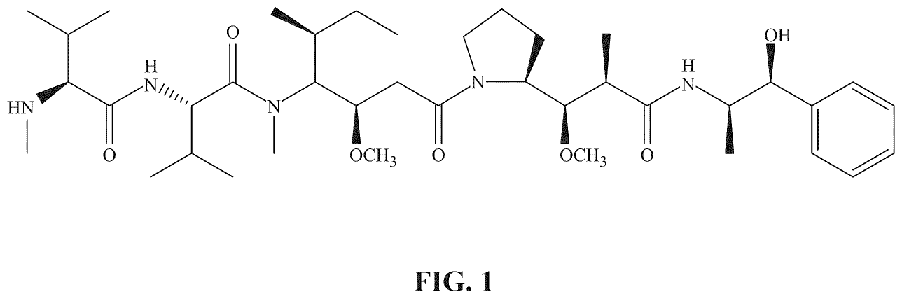

FIG. 2 is schematic diagram of exemplary structures of an antibody-drug conjugate (ADC) of the general structural formula Ab-(L-U)ₙ of the present disclosure under one potential set of conjugation conditions (L is linked to one or more interchain disulfide bond sites of the antibody through sulfhydryl conjugation), wherein n is 1, 2, 3, 4, 5, 6, 7, and 8, respectively, L is Maleimido-Caproyl-Valine-Citrulline-p-Aminobenzyloxy (mc-vc-pAB), U is MMAE, and the structure of “-L-U” is as follows:

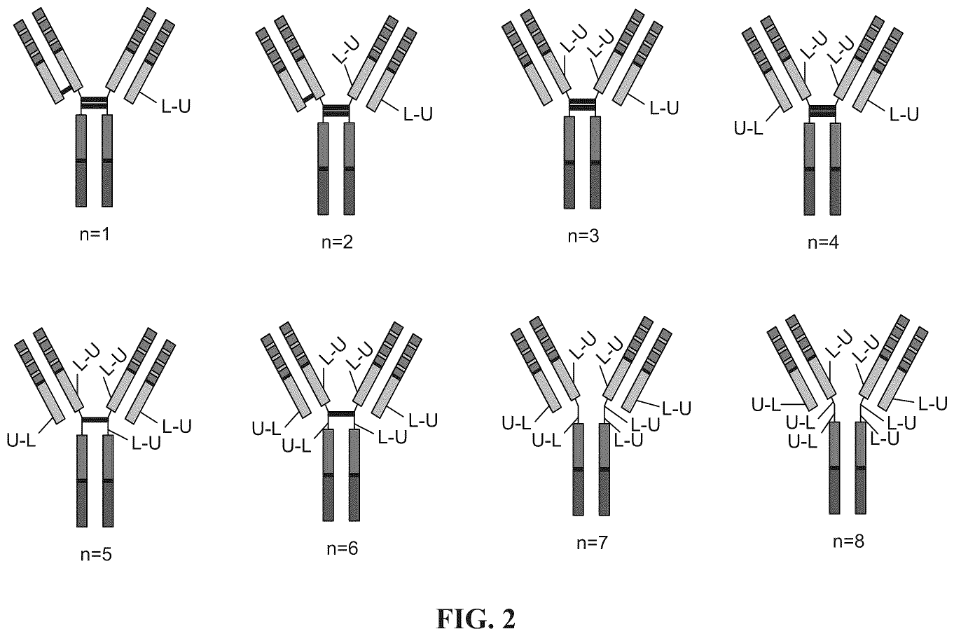

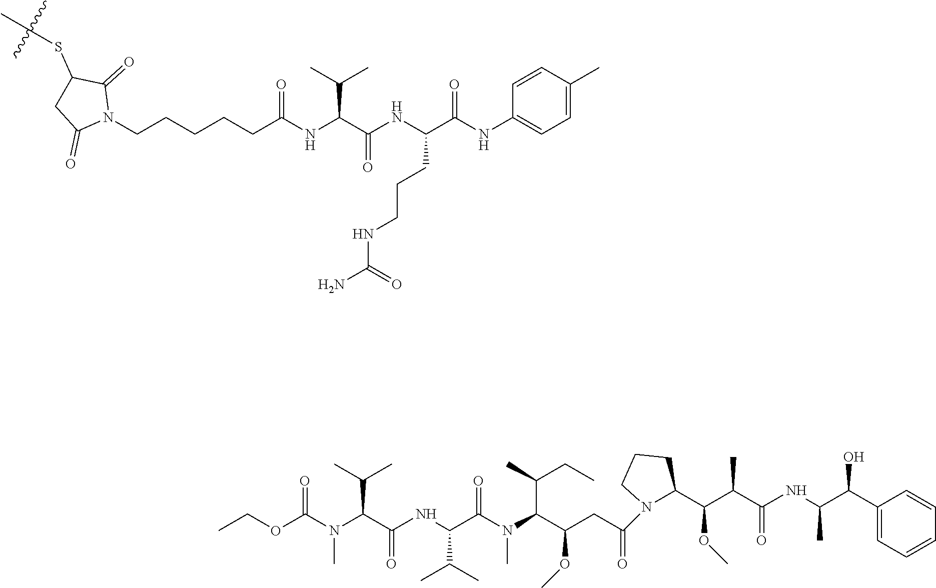

FIG. 3 is a flow chart depicting the evaluation criteria of a HER2 dual-probe in situ hybridization (ISH) test.

### DETAILED DESCRIPTION

The present disclosure provides Human Epidermal Growth Factor Receptor 2 (HER2)-targeting antibody-drug conjugates, as well as methods and uses thereof for the treatment of HER2-low expressing breast cancer. The present disclosure is based, at least in part, on data analysis showing that, surprisingly, a HER2-targeting antibody-drug conjugate (ADC) provided by the present invention (e.g., Disitamab vedotin, i.e. RC48-ADC) showed consistent therapeutic efficacy in HER2 positive and HER2-low expressing subgroups of patients. See, Example 1 herein. The antibody-drug conjugates, methods, and uses provided herein greatly fill the shortage of clinical needs for the treatment of HER2-low expressing breast cancer. Thus, HER2-low expressing breast cancer patients can also benefit significantly from the antibody-drug conjugates (e.g., of RC48-ADC), methods, and uses of the disclosure.

### I. Definitions

Unless otherwise defined, all technical and scientific terms used herein have the same meaning as understood by those of ordinary skill in the art. For definitions and terms in the field, professionals can refer to Current Protocols in Molecular Biology (Ausubel).

The three-letter and one-letter codes for amino acids used in the present disclosure are as described in J. biol. chem, 243, p 3558 (1968).

In the present disclosure, the determination or numbering method of the complementarity determining regions (CDRs) of the variable domains of antibodies includes the IMGT, Kabat, Chothia, AbM, and Contact systems, which are well known in the art.

The term “antibody” as used in the present disclosure encompasses a variety of antibody structures including, but not limited to, monoclonal antibodies, polyclonal antibodies, multispecific antibodies (e.g., bispecific antibodies), and antigen binding fragments. “Antigen binding fragment” as used in the present disclosure refers to an antibody fragment comprising a heavy chain variable region or a light chain variable region of an antibody and being sufficient to retain the same binding specificity as its source antibody and sufficient affinity. In particular, antigen binding fragments comprise Fab, F(ab′), and F(ab′)2, which contain at least one immunoglobulin fragment sufficient to make a specific antigen bind to the polypeptide. The above fragments can be prepared by synthesis, or by an enzymatic method, or by chemical cutting of intact immunoglobulins, or can be genetically engineered by using recombinant DNA techniques. The production methods of the above fragments are well known in the art.

The term “murine antibody” as used in the present disclosure is a monoclonal antibody prepared according to the knowledge and skill in the art. During preparation, a corresponding antigen is injected into the test subjects, and then hybridomas expressing an antibody having the desired sequence or functional characteristics are isolated. In a some embodiments, murine antibodies or antigen binding fragments thereof can further comprise a light chain constant region of murine κ or λ chain or a variant thereof, or further comprise a heavy chain constant region of murine IgG1, IgG2, IgG3, or a variant thereof.

The term “chimeric antibody” as used in the present disclosure is an antibody that is a fusion of a variable region of a murine antibody with a constant region of a human antibody, and can reduce immune responses induced by murine antibodies. When establishing a chimeric antibody, hybridomas which secrete a murine specific monoclonal antibody are first established. Then, variable region genes are cloned from murine hybridoma cells, and as required, constant region genes are cloned from a human antibody. The mouse variable region genes and the human constant region genes are linked to form a chimeric gene and inserted into a human vector Finally, chimeric antibody molecules are expressed in a eukaryotic industrial system or a prokaryotic industrial system. In an embodiment of the disclosure, the antibody light chain of the chimeric antibody further comprises a light chain constant region of human κ or λ chain or a variant thereof. In another embodiment of the disclosure, the antibody heavy chain of the chimeric antibody further comprises a heavy chain constant region of human IgG1, IgG2, IgG3, IgG4, or a variant thereof. The constant region of the human antibody can be selected from the heavy chain constant region of human IgG1, IgG2, IgG3, or IgG4, or a variant thereof. In some embodiments, the constant region of the human antibody is the heavy chain constant region of human IgG2 or IgG4. Alternatively, IgG4 which has no ADCC toxicity (antibody-dependent cell-mediated cytotoxicity) after an amino acid mutation occurred may be used.

The term “humanized antibody” as used in the present disclosure, also known as CDR-grafted antibody, refers to a antibody generated by grafting of a mouse CDR sequence into human antibody variable region framework (i.e., human germline antibody framework sequences of different types). A humanized antibody comprises a CDR region derived from a non-human antibody and the rest of the antibody molecule is derived from one human antibody (or several human antibodies). Furthermore, in order to preserve binding affinity, some residues of the framework region (known as FR) segments can be modified (Jones et al., Nature, 321: 522-525, 1986; Verhoeyen et al., Science, 239: 1534-1536, 1988; and Riechmann et al., Nature, 332: 323-327, 1988). The humanized antibodies or fragments thereof according to the disclosure can be prepared by techniques known to those skilled in the art (e.g., as described in Singer et al., J. Immun. 150: 2844-2857, 1992; Mountain et al., Biotechnol. Genet. Eng. Rev., 10: 1-142, 1992; or Bebbington et al., Bio/Technology, 10: 169-175, 1992).

The term average “DAR” value as used in the present disclosure, namely the Drug-to-Antibody Ratio, refers to the average value of the number of drugs linked to an antibody in an antibody-drug conjugate preparation.

The term “sulfhydryl conjugation” as used in the present disclosure refers to a conjugation means by which a linker is covalently linked to a free sulfhydryl group on an antibody. Cysteine exists in the form of a disulfide bond in the antibody, and there are 4 pairs of interchain disulfide bonds in an IgG antibody, which are easily reduced Therefore, during the preparation of an antibody-drug conjugate, the 4 pairs of interchain disulfide bonds in the IgG antibody are frequently reduced, which produces the above-mentioned free sulfhydryl group on the antibody. Moreover, since there are 4 pairs of interchain disulfide bonds in an IgG antibody, when they are reduced, a maximum of 8 free sulfhydryl groups are generated. An IgG antibody will therefore have a maximum of 8 sulfhydryl conjugation sites. Thus, When n in an antibody-drug conjugate of the general formula Ab-(L-U)ₙ is 1, “L-U” can be covalently linked to any 1 site of the 8 sulfhydryl conjugation sites, similarly, when n is 2, “L-U” can be covalently linked to any 2 sites of the 8 sulfhydryl conjugation sites; when n is 3, “L-U” can be linked to any 3 sites of the 8 sulfhydryl conjugation sites; when n is 4, “L-U” can be covalently linked to any 4 sites of the 8 sulfhydryl conjugation sites; when n is 5, “L-U” can be covalently linked to any 5 sites of the 8 sulfhydryl conjugation sites; when n is 6, “L-U” can be covalently linked to any 6 sites of the 8 sulfhydryl conjugation sites; when n is 7, “L-U” can be covalently linked to any 7 sites of the 8 sulfhydryl conjugation sites; and when n is 8, “L-U” can be covalently linked to the 8 sulfhydryl conjugation sites.

### II. Uses and Methods

Certain aspects of the present disclosure relate to antibody-drug conjugates that bind HER2, as well as to methods and uses of the same.

In some embodiments, the antibody-drug conjugate involved has the structure of the general formula Ab-(L-U)n, wherein Ab represents anti-HER2 (Human Epidermal Growth Factor Receptor 2) antibody; L represents a linker; U represents conjugated cytotoxic molecules; and n is an integer from 1 to 8 (e.g., 1, 2, 3, 4, 5, 6, 7, 8), and represents the number of cytotoxic molecules bound to each antibody.

In some embodiments, the cytotoxic molecule is an auristatin, or an analog or derivative thereof. Auristatins are derivatives of the natural product dolastatin. Exemplary auristatins include dolostatin-10, auristatin E, auristatin T, MMAE (N-methylvaline-valine-dolaisoleuine-dolaproine-norephedrine or monomethyl auristatin E) and MMAF (N-methylvaline-valine-dolaisoleuine-dolaproine-phenylalanine or dovaline-valine-dolaisoleunine-dolaproine-phenylalanine), AEB (ester produced by reacting auristatin E with paraacetyl benzoic acid), AEVB (ester produced by reacting auristatin E with benzoylvaleric acid), and AFP (dimethylvaline-valine-dolaisoleuine-dolaproine-phenylalanine-p-phenylenediamine or auristatin phenylalanine phenylenediamine). WO 2015/057699 describes PEGylated auristatins including MMAE. Additional dolostatin derivatives contemplated for use are disclosed in U.S. Pat. No. 9,345,785, incorporated herein by reference for any purpose.

In some embodiments, the cytotoxic molecule is MMAE. In other embodiments, the cytotoxic agent is MMAF.

In some embodiments, the anti-HER2 (Human Epidermal Growth Factor Receptor 2) antibody or the functional fragment thereof in the antibody-drug conjugate provided by the present disclosure comprises a heavy chain variable region and a light chain variable region, wherein the CDRs of the heavy chain variable region and/or the CDRs of the light chain variable region have the same CDR sequences as Disitamab vedotin; the linker L comprises Maleimido-Caproyl-Valine-Citrulline-p-Aminobenzyloxy (mc-vc-pAB); and the cytotoxic molecules U comprise MMAE (monomethyl auristatin E).

In some embodiments, the linker L is covalently linked to the antibody by means of sulfhydryl conjugation, and the linking site is the interchain disulfide bond site of the antibody.

In some embodiments, the antibody-drug conjugate of the present disclosure is a mixture of antibody-drug conjugates linked with 2-7 cytotoxic molecules, wherein the average DAR (i.e., Drug-to-Antibody Ratio) value of the antibody-drug conjugates is any number from 2 to 7; more preferably, the average DAR value of the antibody-drug conjugates of the present disclosure is approximately equal to 2, 3, 4, 5, 6, or 7. In some specific examples of the present disclosure, the average DAR value of the antibody-drug conjugates of the present disclosure is 4±0.5.

In some embodiments, the corresponding CDRs 1-3 of the heavy chain variable region and the light chain variable region of the anti-HER2 antibody involved in the present disclosure are as follows (IMGT numbering):

TABLE 2
Corresponding CDRs 1-3 of the heavy chain
variable region and the light chain variable
region of the anti-HER2 antibody involved in the
present disclosure (IMGT numbering).

| | | |
|---|---|---|
| HCDR1: | GYTFTDYY | SEQ ID NO: 3 |
| HCDR2: | VNPDHGDS | SEQ ID NO: 4 |
| HCDR3: | ARNYLFDH | SEQ ID NO: 5 |
| LCDR1: | QDVGTA | SEQ ID NO: 6 |
| LCDR2: | WAS | SEQ ID NO: 7 |
| LCDR3: | HQFATYT | SEQ ID NO: 8 |

In some embodiments, the corresponding CDRs 1-3 of the heavy chain variable region and the light chain variable region of the anti-HER2 antibody involved in the present disclosure are as follows (Kabat numbering):

TABLE 3
Corresponding CDRs 1-3 of the heavy chain
variable region and the light chain variable
region of the anti-HER2 antibody involved in the
present disclosure (Kabat numbering).

| | | |
|---|---|---|
| HCDR1: | DYYIH | SEQ ID NO: 11 |
| HCDR2: | RVNPDHGDSYYNQKFKD | SEQ ID NO: 12 |
| HCDR3: | ARNYLFDHW | SEQ ID NO: 13 |
| LCDR1: | KASQDVGTAVA | SEQ ID NO: 14 |
| LCDR2: | WASIRHT | SEQ ID NO: 15 |
| LCDR3: | HQFATYT | SEQ ID NO: 16 |

In some embodiments, the anti-HER2 antibody comprises the corresponding CDRs 1-3 of the heavy chain variable regions and the light chain variable region represented by SEQ ID NOs: 3-8, but with 1, 2, or 3 substitutions (e.g., conservative substitutions), insertions, or deletions relative to SEQ ID NOs: 3-8, but an anti-HER2 antibody comprising that sequence retains the ability to bind to HER2. In some embodiments, the anti-HER2 antibody comprises the corresponding CDRs 1-3 of the heavy chain variable regions and the light chain variable region represented by SEQ ID NOs: 11-16, but with 1, 2, or 3 substitutions (e.g., conservative substitutions), insertions, or deletions relative to SEQ ID NOs: 11-16, but an anti-HER2 antibody comprising that sequence retains the ability to bind to HER2.

In some embodiments, the anti-HER2 (Human Epidermal Growth Factor Receptor 2) antibody in the antibody-drug conjugate provided by the present disclosure is murine, chimeric, humanized or fully human, preferably a humanized monoclonal antibody. In some embodiments, the antibody is a monoclonal antibody.

In some embodiments, the anti-HER2 (Human Epidermal Growth Factor Receptor 2) antibody in the antibody-drug conjugate provided by the present disclosure is IgG, including IgG1, IgG2, IgG3, and IgG4, and more preferably IgG1, IgG2, and IgG4.

In some embodiments, the anti-HER2 antibody comprises a heavy chain variable (VH) region and a light chain variable (VL) region; wherein the VH region comprises an amino acid sequence with at least 90%, at least 91%, at least 92%, at least 93%, at least 94%, at least 95%, at least 96%, at least 97%, at least 98%, at least 99%, or 100% identity to the sequence EVQLVQSGAEVKKPGATVKISCKVSGYTFTDYYIHWVQQAPGKGLEWMGRVNPDHGDSY YNQKFKDKATITADKSTDTAYMELSSLRSEDTAVYFCARNYLFDHWGQGTLVTVSS (SEQ ID NO:9); and/or wherein the VL region comprises an amino acid sequence with at least 90%, at least 91%, at least 92%, at least 93%, at least 94%, at least 95%, at least 96%, at least 97%, at least 98%, at least 99%, or 100% identity to the sequence DIQMTQSPSSVSASVGDRVTITCKASQDVGTAVAWYQQKPGKAPKLLIYWASIRHTGVPSR FSGSGSGTDFTLTISSLQPEDFATYYCHQFATYTFGGGTKVEIK (SEQ ID NO: 10). In certain embodiments, the VH sequence (e.g., having at least 90%, 91%, 92%, 93%, 94%, 95%. 96%, 97%, 98%, or 99% identity to SEQ ID NO:9) contains substitutions (e.g., conservative substitutions), insertions, or deletions relative to SEQ ID NO:9, but an anti-HER2 antibody comprising that sequence retains the ability to bind to HER2. In certain embodiments, a total of 1 to 10 amino acids have been substituted, inserted and/or deleted in SEQ ID NO: 9. In certain embodiments, substitutions, insertions, or deletions occur in regions outside the CDRs (i.e., in the FRs). In certain embodiments, the VL sequence (e.g., having at least 90%, 91%, 92%, 93%, 94%, 95%, 96%, 97%, 98%, or 99% identity to SEQ ID NO:10) contains substitutions (e.g., conservative substitutions), insertions, or deletions relative to SEQ ID NO:10, but an anti-HER2 antibody comprising that sequence retains the ability to bind to HER2. In certain embodiments, a total of 1 to 10 amino acids have been substituted, inserted and/or deleted in SEQ ID NO: 10. In certain embodiments, substitutions, insertions, or deletions occur in regions outside the CDRs (i.e., in the FRs).

In some embodiments, the antibody comprises a heavy chain variable (VH) region and a light chain variable (VL) region; wherein the VH region comprises the amino acid sequence of EVQLVQSGAEVKKPGATVKISCKVSGYTFTDYYIHWVQQAPGKGLEWMGRVNPDHGDSY YNQKFKDKATITADKSTDTAYMELSSLRSEDTAVYFCARNYLFDHWGQGTLVTVSS (SEQ ID NO:9); and wherein the VL region comprises the amino acid sequence of DIQMTQSPSSVSASVGDRVTITCKASQDVGTAVAWYQQKPGKAPKLLIYWASIRHTGVPSR FSGSGSGTDFTLTISSLQPEDFATYYCHQFATYTFGGGTKVEIK (SEQ ID NO:10).

In some embodiments, the heavy chain amino acid sequence of the antibody Ab in the antibody-drug conjugate involved in the present disclosure is shown in SEQ ID NO: 1, and the light chain amino acid sequence thereof is shown in SEQ ID NO: 2. In some embodiments, the heavy chain comprises the amino acid sequence of SEQ ID NO:1 without the C-terminal lysine.

### Heavy Chain Amino Acid Sequence—SEQ ID NO: 1

| | |
|---|---|
| EVQLVQSGAE VKKPGATVKI SCKVSGYTFT DYYIHWVQQA PGKGLEWMGR | 50 |
| VNPDHGDSYY NQKFKDKATI TADKSTDTAY MELSSLRSED TAVYFCARNY | 100 |
| LFDHWGQGTL VTVSSASTKG PSVFPLAPSS KSTSGGTAAL GCLVKDYFPE | 150 |
| PVTVSWNSGA LTSGVHTFPA VLQSSGLYSL SSVVTVPSSS LGTQTYICNV | 200 |
| NHKPSNTKVD KKVEPKSCDK THTCPPCPAP ELLGGPSVFL FPPKPKDTLM | 250 |
| ISRTPEVTCV VVDVSHEDPE VKFNWYVDGV EVHNAKTKPR EEQYNSTYRV | 300 |
| VSVLTVLHQD WLNGKEYKCK VSNKALPAPI EKTISKAKGQ PREPQVYTLP | 350 |
| PSREEMTKNQ VSLTCLVKGF YPSDIAVEWE SNGQPENNYK TTPPVLDSDG | 400 |
| SFFLYSKLTV DKSRWQQGNV FSCSVMHEAL HNHYTQKSLS LSPGK | 445 |

### Light Chain Amino Acid Sequence—SEQ ID NO: 2

| | |
|---|---|
| DIQMTQSPSS VSASVGDRVT ITCKASQDVG TAVAWYQQKP GKAPKLLIYW | 50 |
| ASIRHTGVPS RFSGSGSGTD FTLTISSLQP EDFATYYCHQ FATYTFGGGT | 100 |
| KVEIKRTVAA PSVFIFPPSD EQLKSGTASV VCLLNNFYPR EAKVQWKVDN | 150 |
| ALQSGNSQES VTEQDSKDST YSLSSTLTLS KADYEKHKVY ACEVTHQGLS | 200 |
| SPVTKSFNRG EC | 212 |

In some embodiments, the antibody-drug conjugate of the present disclosure is Disitamab vedotin (e.g., RC48-ADC), which is an antibody-drug conjugate targeting a HER2 target, wherein the linker moiety L is Maleimido-Caproyl-Valine-Citrulline-p-Aminobenzyloxy (mc-vc-pAB); the cytotoxic molecules U comprise MMAE (monomethyl auristatin E); the linker L is covalently linked to the antibody by means of sulfhydryl conjugation; and the average DAR value is 4±0.5.

In some embodiments, the breast cancer involved in the present disclosure (e.g., for treatment according to the present disclosure) is HER2 expression-positive breast cancer, preferably infiltrating locally advanced or metastatic breast cancer as established by histology and/or cytology, and is unresectable.

In some embodiments, the breast cancer involved in the present disclosure (e.g., for treatment according to the present disclosure) is HER2-low expressing breast cancer. Thus, in some embodiments, the patients involved in the present disclosure (e.g., for treatment according to the present disclosure) are HER2-low expressing breast cancer patients. In some embodiments, a HER2-low expressing breast cancer (e.g., in a patient) according to the present disclosure is detected as immunohistochemistry (IHC) 2+/fluorescence in situ hybridization (FISH) negative or IHC1+, e.g., in a sample from the breast cancer. In some embodiments, a HER2-low expressing breast cancer (e.g., in a patient) according to the present disclosure is detected as IHC 2+/FISH negative or IHC1+, e.g., in a sample from the breast cancer.

In some embodiments, HER2 is detected and/or assessed using any suitable method known in the art. For example, HER2 may be detected and/or assessed using an immunohistochemistry (IHC) assay and/or a fluorescence in situ hybridization (FISH) assay. Exemplary methods for detection and assessment of HER2 that may be used in according to the present disclosure are provided below.

Detection and assessment of HER2 may be performed using a variety of samples/specimens. For example, sources of tumor samples/specimens for use according to the present disclosure include, but are not limited to: 1) Surgical resection specimens; 2) Biopsy specimens: and/or 3) Cytological specimens with more than 100 cancer cells.

Samples/specimens for use according to the present disclosure may be processed according to known methods and techniques in the art, for example, using one or more, or all, of the steps of:
(1) immersing specimens immediately after isolation into a standard fixative solution equivalent to 8-10 times the volume of the specimen and fixing using 10% neutral buffered formalin fixative (some large specimens may need to be cut and fixed);
(2) fixing using a fixation time of 6 to 72 h at room temperature; and
(3) wax block embedding, e.g., by replacing reagents of tissue dehydration and wax impregnation in time to ensure sufficient dehydration and wax impregnation effect.

Detection of HER2 may be performed by FISH, e.g., using one or more, or all, of the following steps:

(1) Selecting a representative wax block of tumor tissue. Section by professional and technical personnel, the section is complete, smooth, of uniform thickness, without affecting the diagnosis of knife mark wrinkles. (Tissue containing calcified particles and other uncontrollable factors are excluded), section thickness: 4-5 μm;

(2) Tissue section pretreatment using either of the following methods:

### Method 1 (Manual Operation):

a) Immerse in xylene and dewaxed twice, 15 minutes each time, and then immerse in 100% ethanol for 5 minutes at room temperature,
b) Rehydrate in 100% ethanol, 85% ethanol and 70% ethanol for 2 minutes respectively at room temperature, then immerse in deionized water at room temperature for 3 minutes,
c) Treatment with 90˜93° C. deionized water for 20 minutes,
d) 1 ml gastric enzyme storage solution (200 mg/mL) is dissolved in 200 ml 0.01 MHCL to obtain gastric enzyme working solution (1 mg/ml); Soak the tissue section in gastric enzyme working solution and incubate at 37° C. for 15-30 minutes (the time depends on the thickness of the tissue, generally about 20 minutes),
e) After digestion by gastric enzymes, then rinse in deionized water for 5 minutes,
f) Dehydrate respectively in 70% ethanol, 85% ethanol and 100% ethanol for 2 minutes at room temperature,
g) After drying, then perform the following hybrid denaturation.

### Method 1 (Fully Automatic):

a) Soak in xylene for dewaxing twice at room temperature for 15 minutes each, and then immerse in 100% ethanol twice for 5 minutes each,
b) Dry tissue section at room temperature,
c) Initialize the system and select program, fill the reagent according to the instrument algorithm,
d) Place the dry slides tissue face upward on the glass shelf, put it in the reaction tank, cover the reaction tank cover, close the machine cover, and run the selected program;

(3) Hybridization apparatus denatured hybridization using the following steps:
a) Drop 10 μL probe mixture into the slide hybridization area, immediately cover the slide and seal the edge with rubber glue,
b) Prepare hybridization machine, covariance condition: 75° C., 5 minutes, hybridization condition: 37° C., 16 h; (be careful to maintain humidity in hybridization instrument);

(4) Glass slide rinsing (need to avoid light operation) using the following steps:
a) Carefully remove the cover glass slide, place the glass slide in a solution of 0.3% NP-40/2×SSC at 73° C., shake for 1˜3 seconds, wash for 2 minutes,
b) Rinse at room temperature in 70% ethanol for 3 minutes;

(5) Counterstaining using the following steps:
a) Naturally dried glass slides in dark;
b) Drop 10 μL DAPI at the hybridization site and immediately cover the cover glass. Put in the dark for 10 to 20 minutes, then observe the glass slides under fluorescence microscope with appropriate filter group.

Assessment of HER2, e.g., in a FISH section, for example, generated as described above, may be performed using any suitable method known in the art. For example, using one or more, or all of the following steps.
(a) Observe whole FISH section under low magnification to preliminarily determine the test quality (such as the normal cell signals of normal tissues in the specimen) and whether there is heterogeneity in HER2 amplification;
(b) Find at least 2 areas of invasive cancer and count at least 20 invasive cancer cells. FISH is not suitable for microinvasive nidus with too few cells;
(c) IHC sections can be used to determine the areas of invasive cancer that may be amplified; and
(d) Observe HER2 and CEP17 signals through a specific channel filter under high magnification (60× or 100× objective), and calculate the signal count and ratio.

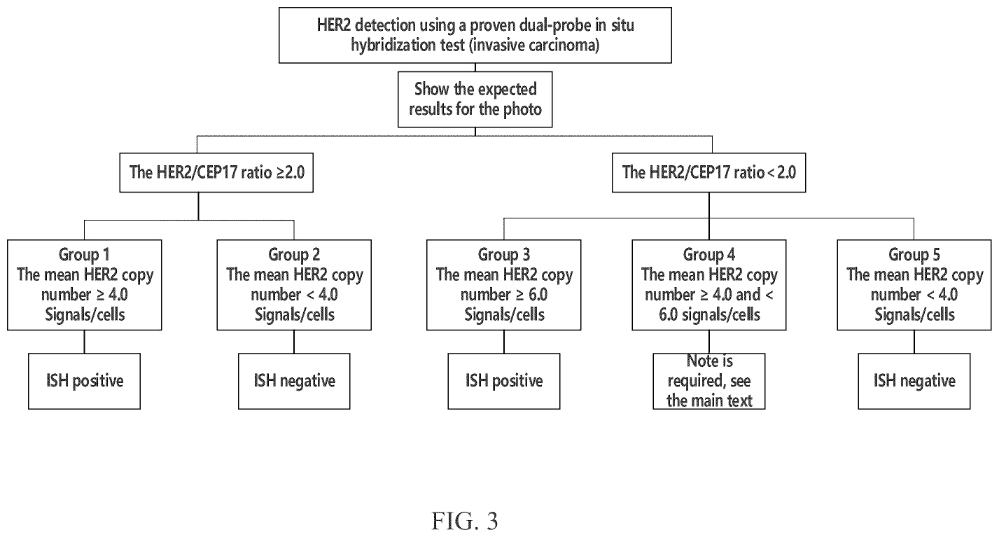

In some embodiments, HER2 is assessed by FISH using dual probes, e.g., using HER2 and CEP17 probes. See, FIG. 3. In some embodiments, assessment of HER2 comprises one or more, or all, of the steps of:
1. Selecting for evaluation tumor cells with consistent nuclear size, intact nuclear borders, homogeneous 4′6-diamidino-2-phenylindole (DAPI) staining, non-overlapping nuclei and clear signals; and
2. Randomly counting at least 20 bicolor signals in invasive cancer nucleus. When observing the signals, the focal length of the microscope is adjusted at any time according to the situation, and the signal located in different planes of the nucleus is accurately observed so as to avoid missing.

In some embodiments, HER2 is assessed according the following criteria (see, also FIG. 3):
(1) Group 1, HER2/CEP17 ratio≥2.0 and mean HER2 copy numbers/cell ratio≥4.0: this situation is evaluated as FISH positive. If many HER2 signals are connected into clusters, it can be directly evaluated as FISH positive.
(2) Group 2, HER2/CEP17 ratio≥2.0 and mean HER2 copy numbers/cell ratio<4.0: it is recommended to increase the number of counted cells for this condition, and if the result remains the same, it is evaluated as FISH negative.
(3) Group 3, HER2/CEP17 ratio<2.0, mean HER2 copy numbers/cell ratio≥6.0: it is recommended to increase the number of counted cells for this condition, and if the results remain unchanged, it is evaluated as FISH positive.
(4) Group 4, HER2/CEP17 ratio<2.0, mean HER2 copy numbers/cell ratio≥4.0 and <6.0: in this condition, it is recommended to recount the signal in at least 20 samples' nuclei, and if the result is different, the two results are analyzed. In such cases, the HER2 status is determined in conjunction with the IHC score, and if the IHC score is 3+, the HER2 status is considered positive. If the IHC score is 0, 1+ or 2+, HER2 status is judged as negative.
(5) Group 5, HER2/CEP17 ratio <2.0, mean HER2 copy numbers/cell ratio<4.0: this condition is evaluated as FISH negative.

In some embodiments, HER2 may be assessed by IHC according to the 2019 Guidelines for The Detection of HER2 in Breast Cancer (Table 4).

TABLE 4
IHC Evaluation criteria of breast cancer HER2.

| | | HER2 |
|---|---|---|
| | IHC | expression |
| Evaluation criteria | score | Evaluation |
| No staining is observed or | 0 | Negative |
| incomplete, faint membrane | | |
| staining is observed in ≤10% | | |
| of invasive cancer cells. | | |
| A faint/barely percetible | 1+ | Negative |
| membrane staining is detected in >10% | | |
| of the invasive cancer cells. | | |
| Weak to moderate complete | 2+ | Uncertain, |
| membrane staining is observed | | further tested |
| in >10% of the invasive cancer cells. | | with in situ |
| Strong and complete membrane | | hybridization or |
| staining is observed in ≤10% | | replace |
| of the invasive cancer cells. | | specimen. |
| Strong, complete and uniform | 3+ | Positive |
| membrane staining is observed | | |
| in >10% of invasive cancer cells. | | |

Evaluation of HER2 by IHC may involve one or more, or all, of the steps of:
1. The entire section is first observed under low magnification to determine whether the staining is satisfactory and whether there is heterogeneity in HER2 expression;
2. Quality control slides are read when evaluating; cytoplasmic and nuclear staining should be negligible, and normal epithelium should not show strong cell membrane staining;
3. Tissue margins and poorly prepared (e.g., obviously extruded) cancer tissue is ignored during evaluation.
4. If the tumor has obvious heterogeneity, the percentage of each scoring level is indicated separately when interpreting.
5. If invasive cancer is the object during evaluation, it is indicated separately if the non-invasive cancer part has overexpressd HER2 (2+ or 3+).
6. If multiple blocks or sections are detected, results are reported separately.

In some embodiments, the patients involved in the present disclosure (e.g., for treatment according to the present disclosure) have previously received one or more prior treatments, including chemotherapy drugs, targeted therapy, immunotherapy and endocrine therapy; preferably, they have previously received taxane systemic therapy; or they have previously received systemic therapy with trastuzumab or a biosimilar thereof at least once.

In some embodiments, the antibody-drug conjugate or medicine of the present disclosure may be administered intranasally, subcutaneously, intradermally, intramuscularly or intravenously. In some embodiments, it is administered at a dose of 2.0 mg/kg every 2 weeks.

### Exemplary Embodiments

Exemplary and non-limiting embodiments of the present disclosure are provided below.

Exemplary embodiment 1: Use of an antibody-drug conjugate (ADC) in the preparation of a medicine for treating of a patient with HER2-low expressing breast cancer, wherein the antibody-drug conjugate has the structure of the general formula Ab-(L-U)ₙ, wherein Ab represents anti-HER2 (Human Epidermal Growth Factor Receptor 2) antibody; L represents a linker; U represents conjugated cytotoxic molecules; and n is an integer from 1 to 8, and represents the number of cytotoxic molecules bound to each antibody, and wherein:
the antibody comprises a heavy chain variable region and a light chain variable region, wherein the CDR of the heavy chain variable region and/or the CDR of the light chain variable region have the same CDR sequences as Disitamab vedotin;
the linker L comprises Maleimido-Caproyl-Valine-Citrulline-p-Aminobenzyloxy (mc-vc-pAB), wherein the linker is covalently linked to the antibody by means of sulfhydryl conjugation, and the linking site is the interchain disulfide bond site of the antibody; and
the cytotoxic molecules U comprise MMAE (monomethyl auristatin E).

Exemplary embodiment 2: The use according to embodiment 1, wherein the HER2-low expressing breast cancer patient is a patient whose HER2 is detected as IHC 2+/FISH negative or IHC1+.

Exemplary embodiment 3: The use according to embodiment 2, wherein the antibody is a murine, chimeric, humanized or fully human antibody.

Exemplary embodiment 4: The use according to embodiment 3, wherein the antibody is IgG, further preferably IgG1, IgG2, and IgG4.

Exemplary embodiment 5: The use according to embodiment 2, wherein the amino acid sequence of the heavy chain of the antibody is shown in SEQ ID NO:1, and the amino acid sequence of the light chain of the antibody is shown in SEQ ID NO:2.

Exemplary embodiment 6: The use according to embodiment 2, wherein the antibody-drug conjugate is Disitamab vedotin.

Exemplary embodiment 7: The use according to embodiment 6, wherein the average DAR (i.e., Drug-to-Antibody Ratio) value of the antibody-drug conjugate is any number from 2 to 7; or more preferably, the average DAR value is 4=0.5.

Exemplary embodiment 8: The use according to embodiment 2, wherein the breast cancer is infiltrating locally advanced or metastatic breast cancer as established by histology and/or cytology, and is unresectable.

Exemplary embodiment 9: The use according to embodiment 2, wherein the patient has previously received one or more prior treatments, including chemotherapy drugs, targeted therapy, immunotherapy and endocrine therapy.

Exemplary embodiment 10 The use according to embodiment 8, wherein the patient has previously received taxane systemic therapy.

Exemplary embodiment 11: The use according to embodiment 8, wherein the patient has previously received systemic therapy with trastuzumab or a biosimilar thereof at least once.

Exemplary embodiment 12: The use according to embodiment 3, wherein the medicine is administered intranasally, subcutaneously, intradermally, intramuscularly or intravenously.

Exemplary embodiment 13: The use according to embodiment 3, wherein the antibody-drug conjugate is administered at a dose of 2.0 mg/kg every 2 weeks.

### EXAMPLES

The examples below are not intended to limit the scope of the present disclosure. The experimental methods not specified for the specific conditions in the following examples are selected according to conventional methods and conditions, or according to the product instructions.

### Example 1: Disitamab Vedotin (RC48 ADC) in HER2-Positive and HER2-Low Expressing Advanced Breast Cancer Patients, a Pooled Analysis of Two Clinical Studies (NCT02881138; NCT03052634)

This Example describes a pooled analysis of two studies (C001 CANCER [NCT02881138] and C003 CANCER [NCT03052634]) for the efficacy and safety of RC48-ADC in HER2-positive or HER2-low expressing advanced breast cancer patients.

C001 CANCER (NCT02881138) is a dose-escalation phase 1 study (0.5, 1.0, 1.5, 2.0 and 2.5 mg/kg) with HER2 positive patients in a 3+3 design.

C003 CANCER (NCT03052634) is a phase Ib study with 1.5, 2.0, 2.5 mg/kg dose being used in the HER2-positive subgroup and 2.0 mg/kg dose being used in both of IHC 2+/FISH− and IHC 1+ HER2-low expressing subgroups. C003 CANCER is currently in progress for patients with IHC 1+ or higher.

A pooled analysis of these two studies for the efficacy and safety of RC48-ADC in HER2-positive or HER2-low expressing subgroups was performed.

### Methods

Detection and assessment of HER2 was performed using surgical resection specimens, biopsy specimens, or cytological specimens with more than 100 cancer cells.

Specimens were processed by:
(1) Immersing specimens immediately after isolation into a standard fixative solution equivalent to 8-10 times the volume of the specimen and fixing using 10% neutral buffered formalin fixative (some large specimens were cut and fixed);
(2) Fixing using a fixation time of 6 to 72 h at room temperature; and
(3) Wax block embedding by replacing reagents of tissue dehydration and wax impregnation in time to ensure sufficient dehydration and wax impregnation effect.

Detection of HER2 was performed by fluorescence in situ hybridization (FISH) assay using the following steps:
(1) Selecting a representative wax block of tumor tissue. Sections were done by professional and technical personnel, the sections were complete, smooth, of uniform thickness, without affecting the diagnosis of knife mark wrinkles. (Tissue containing calcified particles and other uncontrollable factors were excluded), section thickness: 4-5 μm;
(2) Tissue sections were pretreated using either of the following methods:

### Method 1 (Manual Operation):

a) Sections were immersed in xylene and dewaxed twice, 15 minutes each time, and then immersed in 100% ethanol for 5 minutes at room temperature,
b) Sections were rehydrated in 100% ethanol, 85% ethanol and 70% ethanol for 2 minutes respectively at room temperature, then immersed in deionized water at room temperature for 3 minutes,
c) Sections were treated with 90˜93° C. deionized water for 20 minutes,
d) 1 ml gastric enzyme storage solution (200 mg/mL) was dissolved in 200 ml 0.01 MHCL to obtain gastric enzyme working solution (1 mg/ml); The tissue sections were soaked in gastric enzyme working solution and incubated at 37° C. for 15-30 minutes (the time depended on the thickness of the tissue, generally about 20 minutes),
e) After digestion by gastric enzymes, the sections were rinsed in deionized water for 5 minutes,
f) Sections were dehydrated respectively in 70% ethanol, 85% ethanol and 100% ethanol for 2 minutes at room temperature,
g) After drying, the following hybrid denaturation was performed.

### Method 2 (Fully Automatic):

a) Sections were soaked in xylene for dewaxing twice at room temperature for 15 minutes each, and then immersed in 100% ethanol twice for 5 minutes each,
b) Tissue sections were dried at room temperature,
c) The system was initialized and program was selected, the reagent was filled according to the instrument algorithm,
d) Dry slides were placed tissue face upward on the glass shelf, put it in the reaction tank, the reaction tank was covered, the machine cover was closed, and the selected program was run;

(3) Denatured hybridization was performed using the following steps:
a) 10 μL probe mixture was dropped into the slide hybridization area, the slide was immediately covered and the edge was sealed with rubber glue,
b) The hybridization machine was prepared, covariance condition: 75° C., 5 minutes, hybridization condition: 37° C., 16 h; (being careful to maintain humidity in hybridization instrument);

(4) Glass slides were rinsed (needing to avoid light operation) using the following steps
a) The cover glass slide was carefully removed, the glass slide was placed in a solution of 0.3% NP-40/2×SSC at 73° C., shaken for 1-3 seconds, washed for 2 minutes, followed by rinsing at room temperature in 70% ethanol for 3 minutes;

(5) Counterstaining was performed using the following steps:
a) Dried glass slides were naturally dried in dark;
b) 10 μL DAPI was dropped at the hybridization site and the cover glass was immediately covered, followed by putting in the dark for 10 to 20 minutes. The glass slides were observed under fluorescence microscope with appropriate filter group.

Assessment of HER2 was performed using the following steps.
(a) Whole FISH sections were observed under low magnification to preliminarily determine the test quality (such as the normal cell signals of normal tissues in the specimen) and whether there was heterogeneity in HER2 amplification,
(b) At least 2 areas of invasive cancer were found and at least 20 invasive cancer cells were counted. FISH is not suitable for microinvasive nidus with too few cells,
(c) IHC sections were used to determine the areas of invasive cancer that may be amplified, and
(d) HER2 and CEP17 signals were observed through a specific channel filter under high magnification (60× or 100× objective), and the signal count and ratio were calculated.

HER2 was assessed by FISH using dual probes as follows (see. FIG. 3):
1. Tumor cells with consistent nuclear size, intact nuclear borders, homogeneous 4′6-diamidino-2-phenylindole (DAPI) staining, non-overlapping nuclei and clear signals were selected for evaluation; and
2. At least 20 bicolor signals in invasive cancer nucleus were randomly counted. When observing the signals, the focal length of the microscope was adjusted at any time according to the situation, and the signal located in different planes of the nucleus was accurately observed so as to avoid missing.

HER2 was assessed according the following criteria (see, also FIG. 3):
(1) Group 1, HER2/CEP17 ratio≥2.0 and mean HER2 copy numbers/cell ratio≥4.0: this situation was evaluated as FISH positive. If many HER2 signals were connected into clusters, it was directly evaluated as FISH positive.
(2) Group 2, HER2/CEP17 ratio≥2.0 and mean HER2 copy numbers/cell ratio<4.0: the number of counted cells was increased for this condition, and if the result remained the same, it was evaluated as FISH negative.
(3) Group 3, HER2/CEP17 ratio<2.0, mean HER2 copy numbers/cell ratio≥6.0: the number of counted cells was increased for this condition, and if the results remained unchanged, it was evaluated as FISH positive.
(4) Group 4, HER2/CEP17 ratio<2.0, mean HER2 copy numbers/cell ratio≥4.0 and <6.0: in this condition, the signal was recounted in at least 20 samples' nuclei, and if the result was different, the two results were analyzed. In such cases, the HER2 status was determined in conjunction with the IHC score, and if the IHC score was 3+, the HER2 status was considered positive. If the IHC score was 0, 1+ or 2+, HER2 status was judged as negative.
(5) Group 5, HER2/CEP17 ratio<2.0, mean HER2 copy numbers/cell ratio<4.0: this condition was evaluated as FISH negative.

HER2 was assessed by IHC according to the 2019 Guidelines for The Detection of HER2 in Breast Cancer (Table 5).

TABLE 5
IHC Evaluation criteria of breast cancer HER2.

| | | HER2 |
|---|---|---|
| | IHC | expression |
| Evaluation criteria | score | Evaluation |
| No staining is observed or | 0 | Negative |
| incomplete, faint membrane staining is | | |
| observed in ≤10% of invasive cancer cells. | | |
| A faint/barely percetible membrane | 1+ | Negative |
| staining is detected in >10% of the | | |
| invasive cancer cells. | | |
| Weak to moderate complete membrane | 2+ | Uncertain, |
| staining is observed in >10% of the | | further tested |
| invasive cancer cells. | | |
| Strong and complete membrane staining | | with in situ |
| is observed in ≤10% of the | | hybridization or |
| invasive cancer cells. | | replace |
| | | specimen. |
| Strong, complete and uniform | 3+ | Positive |
| membrane staining is observed | | |
| in >10% of invasive cancer cells. | | |

Evaluation of HER2 by IHC was performed as follows:
1. The entire section was first observed under low magnification to determine whether the staining was satisfactory and whether there was heterogeneity in HER2 expression;
2. Quality control slides were read when evaluating; cytoplasmic and nuclear staining should be negligible, and normal epithelium should not show strong cell membrane staining;
3. Tissue margins and poorly prepared (e.g., obviously extruded) cancer tissue were ignored during evaluation.
4. If the tumor had obvious heterogeneity, the percentage of each scoring level was indicated separately when interpreting.
5. If invasive cancer was the object during evaluation, it was indicated separately if the non-invasive cancer part had overexpressd HER2 (2+ or 3+).
6. If multiple blocks or sections were detected, results were reported separately.

### Results

At the data cutoff date (Dec. 31, 2020), 118 female breast cancer patients were enrolled and treated with RC48-ADC. 70 patients (59.3%) were HER2-positive, and 48 patients (40.7%) were HER2-low expressing. At baseline, 77 patients (65.3%) had liver metastases, 50 patients (42.4%) were at Eastern Cooperative Oncology Group (ECOG) Performance Status (PS) 1, and 47 patients (39.8%) had received 3 prior chemotherapy regimens.

In the HER2-positive subgroups, the objective remission rate (ORR) were 22.2% (95% confidence interval [CI]: 6.4%, 47.6%), 42.9% (95% CI: 21.8%, 66.0%), and 40.0% (95% CI: 21.1%, 61.3%) for the 1.5, 2.0, and 2.5 mg/kg doses, respectively. The median progression free survival (mPFS) was 4.0 months (95% CI: 2.6, 7.6), 5.7 months (95% CI: 5.3, 8.4) and 6.3 months (95% CI: 4.3, 8.8) for the 1.5, 2.0 and 2.5 mg/kg cohorts.

In the HER2-low expressing subgroups, the ORR and mPFS were 39.6% (95% CI: 25.8%, 54.7%) and 5.7 months (95% CI: 4.1, 8.3), respectively. The ORR and mPFS of IHC2+/FISH patients were 42.9% (15/35) and 6.6 months (95% CI: 4.1, 8.5), respectively. For IHC1+ patients, even though the COVID-19 pandemic caused some patients to delay treatment, the ORR and mPFS reached 30.8% (4/13) and 5.5 months (95% CI: 2.7, 11.0), respectively.

Common treatment-related adverse events (TRAEs) were as follows increased AST (64.4%), increased ALT (59.3%), hypoesthesia (58.5%), decreased white blood cell count (48.3%), and decreased neutrophil count (47.5%), and most were at a severity of grade 1-2. The subjects whose neutrophil counts decreased by 3 grades (16.9%), had increased gamma glutamyl transferase (GGT; 12.7%) and had fatigue (11.9%) higher than TRAE accounted for 10% of the total population.

### CONCLUSIONS

RC48-ADC showed consistent efficacy in HER2-positive and HER2-low expressing subgroups. This showed a more favorable benefit-risk ratio at 2.0 mg/kg once every 2 weeks (Q2W) compared to other dose levels.

The invention has been exemplified by specific examples. However, those skilled in the art will appreciate that the present invention is not limited to the specific embodiments. Various modifications or variations can be made within the scope of the present disclosure, and various technical features mentioned throughout the present specification can be combined with each other without deviating from the spirit and scope of the present disclosure. Such modifications and variations are all within the scope of the present disclosure.

1.-23. (canceled)

24. A method for treating a patient with Human Epidermal Growth Factor Receptor 2 (HER2)-low expressing breast cancer, comprising administering to the patient a therapeutically effective amount of an antibody-drug conjugate (ADC), wherein the ADC has the structure of the general formula Ab-(L-U)ₙ, wherein:
Ab represents an anti-HER2 antibody,
L represents a linker,
U represents a conjugated cytotoxic molecule, and
n is an integer from 1 to 8 and represents the number of cytotoxic molecules bound to each antibody;
wherein the antibody comprises a heavy chain variable region and a light chain variable region, wherein the CDR sequences of the heavy chain variable region and/or the CDR sequences of the light chain variable region have the same CDR sequences as Disitamab vedotin;
wherein the linker L comprises Maleimido-Caproyl-Valine-Citrulline-p-Aminobenzyloxy (mc-vc-pAB), wherein the linker is covalently linked to the anti-HER2 antibody by means of sulfhydryl conjugation, and the linking site is the interchain disulfide bond site of the anti-HER2 antibody; and
wherein the cytotoxic molecule U comprises MMAE (monomethyl auristatin E).

25. The method of claim 24, wherein the HER2-low expressing breast cancer patient is a patient whose HER2 is detected as immunohistochemistry (IHC) 2+/fluorescence in situ hybridization (FISH) negative or IHC1+.

26. The method of claim 25, wherein HER2 is detected as IHC 2+/FISH negative or IHC1+ in a sample from the breast cancer.

27. The method of claim 24, wherein HER2 is detected using an immunohistochemistry (IHC) assay and/or a fluorescence in situ hybridization (FISH) assay.

28. The method of claim 24, wherein the anti-HER2 antibody is a murine, chimeric, humanized or fully human antibody.

29. The method of claim 28, wherein the anti-HER2 antibody is of the IgG class.

30. The method of claim 29, wherein the anti-HER2 antibody has an IgG1, IgG2, or IgG4 isotype.

31. The method of claim 24, wherein the anti-HER2 antibody comprises a heavy chain variable region (VH) and a light chain variable region (VL), wherein:
(a) the VH comprises a CDR-H1 comprising the amino acid sequence GYTFTDYY (SEQ ID NO:3), a CDR-H2 comprising the amino acid sequence VNPDHGDS (SEQ ID NO:4), and a CDR-H3 comprising the amino acid sequence ARNYLFDH (SEQ ID NO:5), and
(b) the VL comprises a CDR-L1 comprising the amino acid sequence QDVGTA (SEQ ID NO:6), a CDR-L2 comprising the amino acid sequence WAS (SEQ ID NO:7), and a CDR-L3 comprising the amino acid sequence HQFATYT (SEQ ID NO:8).

32. The method of claim 24, wherein the anti-HER2 antibody comprises a heavy chain variable region (VH) and a light chain variable region (VL), wherein:
(a) the VH comprises a CDR-H1 comprising the amino acid sequence DYYIH (SEQ ID NO: 11), a CDR-H2 comprising the amino acid sequence RVNPDHGDSYYNQKFKD (SEQ ID NO: 12), and a CDR-H3 comprising the amino acid sequence ARNYLFDHW (SEQ ID NO: 13), and
(b) the VL comprises a CDR-L1 comprising the amino acid sequence KASQDVGTAVA (SEQ ID NO: 14), a CDR-L2 comprising the amino acid sequence WASIRHT (SEQ ID NO: 15), and a CDR-L3 comprising the amino acid sequence HQFATYT (SEQ ID NO: 16).

33. The method of claim 24, wherein the anti-HER2 antibody comprises a heavy chain variable region (VH) comprising the amino acid sequence of SEQ ID NO: 9, and a light chain variable region (VL) comprising the amino acid sequence of SEQ ID NO: 10.

34. The method of claim 24, wherein the anti-HER2 antibody is a human IgG antibody.

35. The method of claim 34, wherein the anti-HER2 antibody is a human IgG1, IgG2, IgG3, or IgG4 antibody.

36. The method of claim 24, wherein the amino acid sequence of the heavy chain of the antibody is shown in SEQ ID NO:1, and the amino acid sequence of the light chain of the antibody is shown in SEQ ID NO:2.

37. The method of claim 24, wherein the ADC is Disitamab vedotin or a biosimilar thereof.

38. The method of claim 24, wherein the average Drug-to-Antibody Ratio (DAR) value of the ADC is any number from 2 to 7.

39. The method of claim 38, wherein the average DAR value is 4±0.5.

40. The method of claim 24, wherein the breast cancer is infiltrating locally advanced or metastatic breast cancer as established by histology and/or cytology, and is unresectable.

41. The method of claim 24, wherein the patient has previously received one or more prior treatments.

42. The method of claim 41, wherein the one or more prior treatments are selected from the group consisting of a chemotherapy drug, a targeted therapy, an immunotherapy and an endocrine therapy.

43. The method of claim 42, wherein the patient has previously received taxane systemic therapy.

44. The method of claim 42, wherein the patient has previously received systemic therapy with trastuzumab or a biosimilar thereof at least once.

45. The method of claim 24, wherein the medicine is administered intranasally, subcutaneously, intradermally, intramuscularly or intravenously.

46. The method of claim 24, wherein the ADC is administered at a dose of 2.0 mg/kg every 2 weeks


SEQUENCE LISTING (16 sequences)

SEQ ID NO: 1 - protein, 445 aa, synthetic construct
  REGION 1..445: note = Synthetic Construct
EVQLVQSGAEVKKPGATVKISCKVSGYTFTDYYIHWVQQAPGKGLEWMGRVNPDHGDSYYNQKFKDKATITADKSTDTAYMELSSLRSEDTAVYFCARNYLFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 212 aa, synthetic construct
  REGION 1..212: note = Synthetic Construct
DIQMTQSPSSVSASVGDRVTITCKASQDVGTAVAWYQQKPGKAPKLLIYWASIRHTGVPSRFSGSGSGTDFTLTISSLQPEDFATYYCHQFATYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 8 aa, synthetic construct
  REGION 1..8: note = Synthetic Construct
GYTFTDYY

SEQ ID NO: 4 - protein, 8 aa, synthetic construct
  REGION 1..8: note = Synthetic Construct
VNPDHGDS

SEQ ID NO: 5 - protein, 8 aa, synthetic construct
  REGION 1..8: note = Synthetic Construct
ARNYLFDH

SEQ ID NO: 6 - protein, 6 aa, synthetic construct
  REGION 1..6: note = Synthetic Construct
QDVGTA

SEQ ID NO: 7 - skipped

SEQ ID NO: 8 - protein, 7 aa, synthetic construct
  REGION 1..7: note = Synthetic Construct
HQFATYT

SEQ ID NO: 9 - protein, 115 aa, synthetic construct
  REGION 1..115: note = Synthetic Construct
EVQLVQSGAEVKKPGATVKISCKVSGYTFTDYYIHWVQQAPGKGLEWMGRVNPDHGDSYYNQKFKDKATITADKSTDTAYMELSSLRSEDTAVYFCARNYLFDHWGQGTLVTVSS

SEQ ID NO: 10 - protein, 105 aa, synthetic construct
  REGION 1..105: note = Synthetic Construct
DIQMTQSPSSVSASVGDRVTITCKASQDVGTAVAWYQQKPGKAPKLLIYWASIRHTGVPSRFSGSGSGTDFTLTISSLQPEDFATYYCHQFATYTFGGGTKVEIK

SEQ ID NO: 11 - protein, 5 aa, synthetic construct
  REGION 1..5: note = Synthetic Construct
DYYIH

SEQ ID NO: 12 - protein, 17 aa, synthetic construct
  REGION 1..17: note = Synthetic Construct
RVNPDHGDSYYNQKFKD

SEQ ID NO: 13 - protein, 9 aa, synthetic construct
  REGION 1..9: note = Synthetic Construct
ARNYLFDHW

SEQ ID NO: 14 - protein, 11 aa, synthetic construct
  REGION 1..11: note = Synthetic Construct
KASQDVGTAVA

SEQ ID NO: 15 - protein, 7 aa, synthetic construct
  REGION 1..7: note = Synthetic Construct
WASIRHT

SEQ ID NO: 16 - protein, 7 aa, synthetic construct
  REGION 1..7: note = Synthetic Construct
HQFATYT